(19) United States
(12) Patent Application Publication
(10) Pub. No.: US 2004/0038388 A1
(43) Pub. Date: Feb. 26, 2004

(54) Manufacturing process for array plate assembly

(75) Inventors: Melvin Yamamoto, Fremont, CA (US); Eric J. Spence, San Jose, CA (US)

Correspondence Address: AFFYMETRIX, INC, ATTN: CHIEF IP COUNSEL, LEGAL DEPT., 3380 CENTRAL EXPRESSWAY, SANTA CLARA, CA, 95051, US

(73) Assignee: Affymetrix, INC., Santa Clara, CA (US)

(21) Appl. No.: 10/428,626

(22) Filed: May 2, 2003

Related U.S. Application Data
(63) Continuation-in-part of application No. 10/325,171, filed on Dec. 19, 2002.
(60) Provisional application No. 60/319,222, filed on May 2, 2002.
(60) Provisional application No. 60/344,084, filed on Dec. 19, 2001.

(51) Int. Cl. C12M 1/34

(52) U.S. Cl. 435/287.2; 435/288.4; 435/6

(57) ABSTRACT

In one embodiment, a method is provided for manufacturing an array plate consisting of several different arrays.

### BACKGROUND OF THE INVENTION

This application claims priority of U.S. Provisional Application Serial No. 60/319,213, filed on May 2, 2002. This application is also a Continuation in part of U.S. patent application No. 10/325,171, filed on Dec. 19, 2003, which is a Non-Provisional of U.S. Provisional Application No. 60/344,084, filed on Dec. 19, 2001. The '213, '171 and '084 applications are incorporated herein by reference for all purposes.

### SUMMARY OF THE INVENTION

One embodiment of the invention relates to methods of making array plates, apparatuses for high throughput screening of biological samples, and systems for performing simultaneously parallel screening of many samples against replicates of the same array as well as parallel screening of many arrays against the same sample.

One embodiment of the present invention provides several methods of making array plates. In one method, a wafer and a body are provided. The wafer includes a substrate and a surface to which is attached a plurality of array probes. The body is a plate which has a plurality of grid-shaped cavities or wells. The wafer is attached to one surface of the body wherein the wafer and the body are held together with adhesive whereby a coat of an adhesive is applied to the body, thereby forming wells defining spaces for receiving samples.

In an embodiment of the invention, the method also includes a curing process of the adhesive by UV light, heat, pressure, air, or any combination of these. In a further embodiment of the invention, the body has a recess in one surface wherein the adhesive is placed. The recess is useful for preventing leakage of the adhesive in the wells defining spaces.

In another method of the invention, a wafer and a body are provided. The body is a plate which has a plurality of grid-shaped cavities or wells. The body includes gaskets or o-rings which are molded onto the body at one surface. The wafer is then mounted to the surface of the body where the gaskets or o-rings are located. A clamping plate is used to hold the wafer and the body together. The clamping plate is attached to the outside perimeter of the body. In certain embodiments, the clamping plate is attached to the outside perimeter of the body by adhesive, screws, ultrasonic welding, or snaps.

In another method of the invention, a body and at least one arrays is provided. The body is a plate which has at least one grid-shaped cavity or well. Each array is captured at the bottom of each well of the body. In certain embodiments of the invention, the individual arrays can be attached to the body by adhesive, screws, ultrasonic welding or snaps.

In another method of the invention, a body and a plurality of arrays are provided. The body is a plate which has a plurality of grid-shaped cavities or wells. Each array is captured at the bottom of each well of the body with a clamping plate. The clamping plate is then attached to the outside perimeter of each well. In certain embodiments of the invention, the clamping plate is attached to the outside perimeter of each well by adhesive, screws, ultrasonic welding or snaps.

In a further method of the invention, a body and a plurality of arrays are provided. The body is a plate which has a plurality of grid-shaped cavities or wells. The arrays are placed on the cavities of the body wherein the walls of the body surrounding the arrays are higher than the thickness of the arrays. A hot plate or ultrasonic horn is used to melt or swag and reform the wall surrounding each array and therefore, capturing the arrays to the body. In one embodiment of the invention, the individual arrays can be captured one at a time or several arrays at once. In another embodiment, a gasket may be attached directly to the body, along the perimeter of the array, inside the high walls. When the walls are melted or swaged by ultrasonic welding, the gasket is compressed against the array, sealing the array in the well.

Other objects, features and advantages of the present invention will become apparent to those of skill in art by reference to the figures, the description that follows and the claims.

### BRIEF DESCRIPTION OF THE DRAWINGS

FIG. 1 depicts a system of this invention having an array plate, fluid handling device, array plate reader and computer;

FIG. 2 depicts the scanning of an array plate by an array plate reader;

FIG. 3 depicts a biological plate;

FIG. 4 depicts the mating of a wafer containing many arrays with a body having channels to create an array plate;

FIG. 5 depicts an array plate in cross section having a body attached to a wafer to create closed wells in which a probe array is exposed to the space in the well;

FIG. 6 depicts a biological plate in cross section having a body which has individual arrays attached to the bottom of the wells;

FIG. 7 is a top-down view of a well containing an array;

FIG. 8 depicts a method of producing an array of oligonucleotide probes on the surface of a substrate by using a mask to expose certain parts of the surface to light, thereby removing photoremovable protective groups, and attaching nucleotides to the exposed reactive groups.

FIG. 9 depicts an embodiment of this invention having a wafer mated with a grid through adhesive bonding.

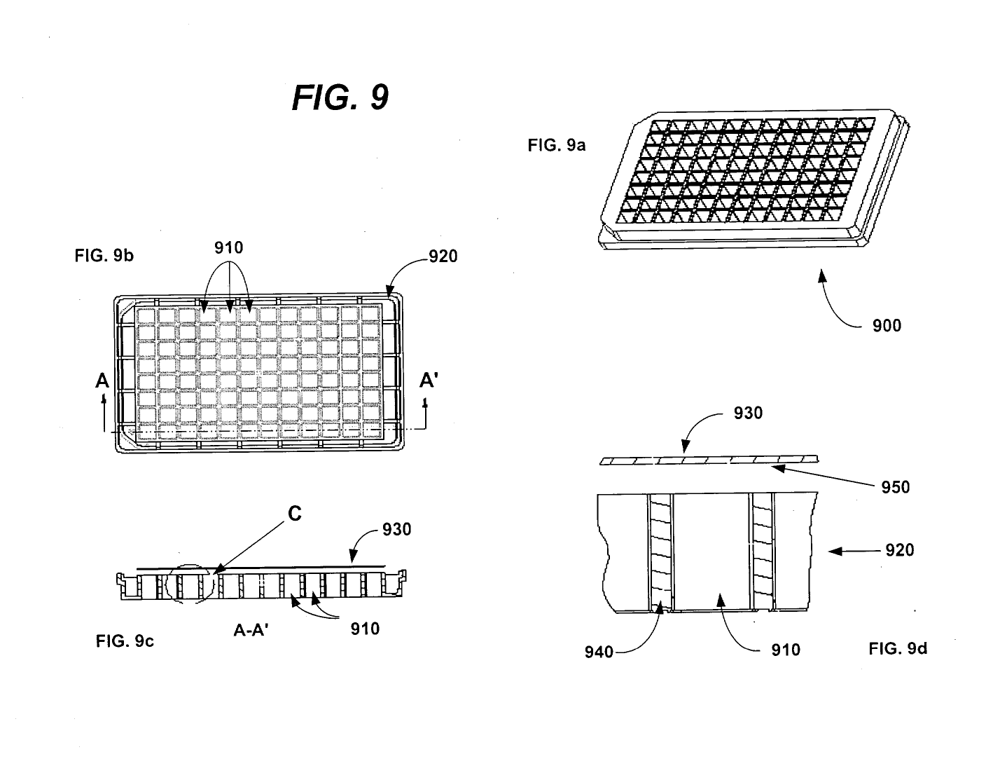

FIG. 10 depicts an embodiment of this invention having a wafer and a grid with a recess and/or gaskets or o-rings wherein adhesive is placed in the recess for attachment of the wafer to the grid.

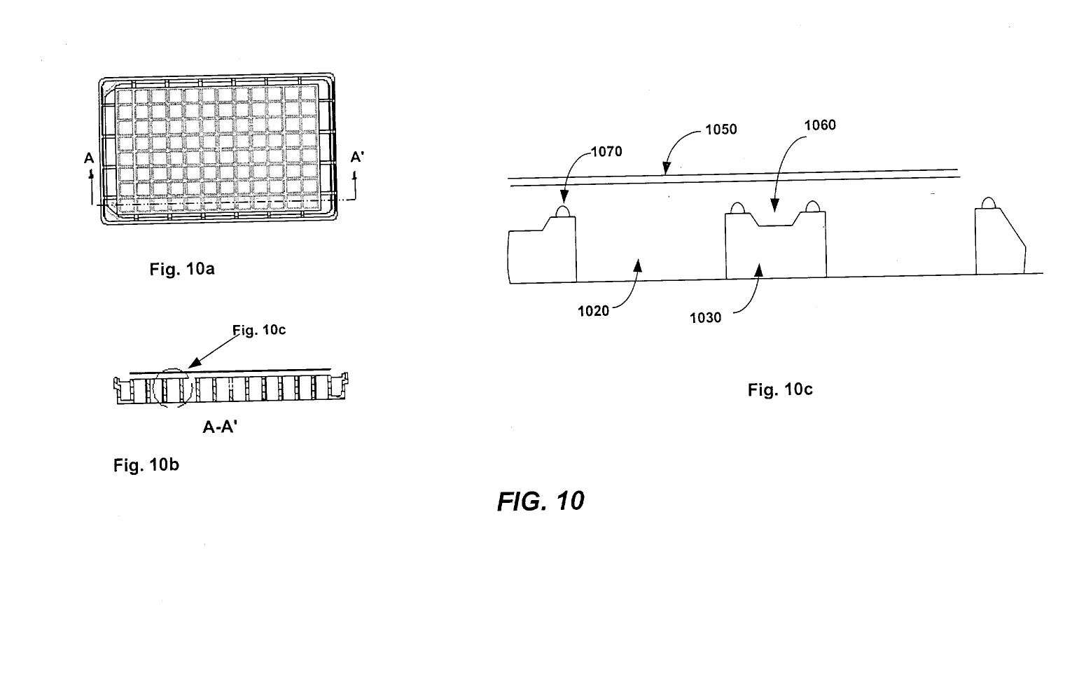

FIG. 11 depicts an embodiment of this invention, wherein the attachment of a wafer with a grid with a clamping plate.

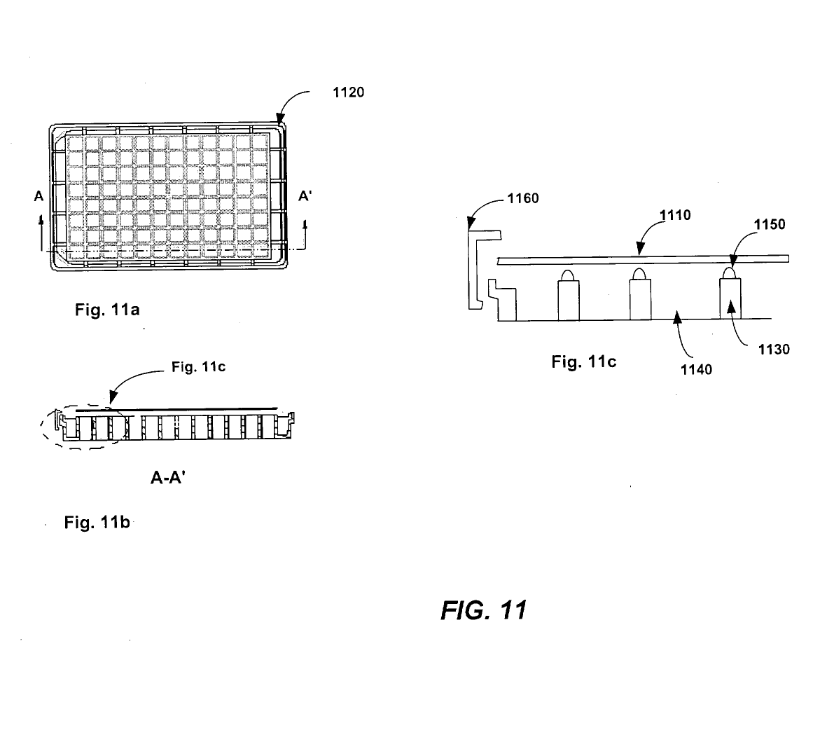

FIG. 12 depicts an embodiment of this invention, wherein individual arrays are captured on a grid.

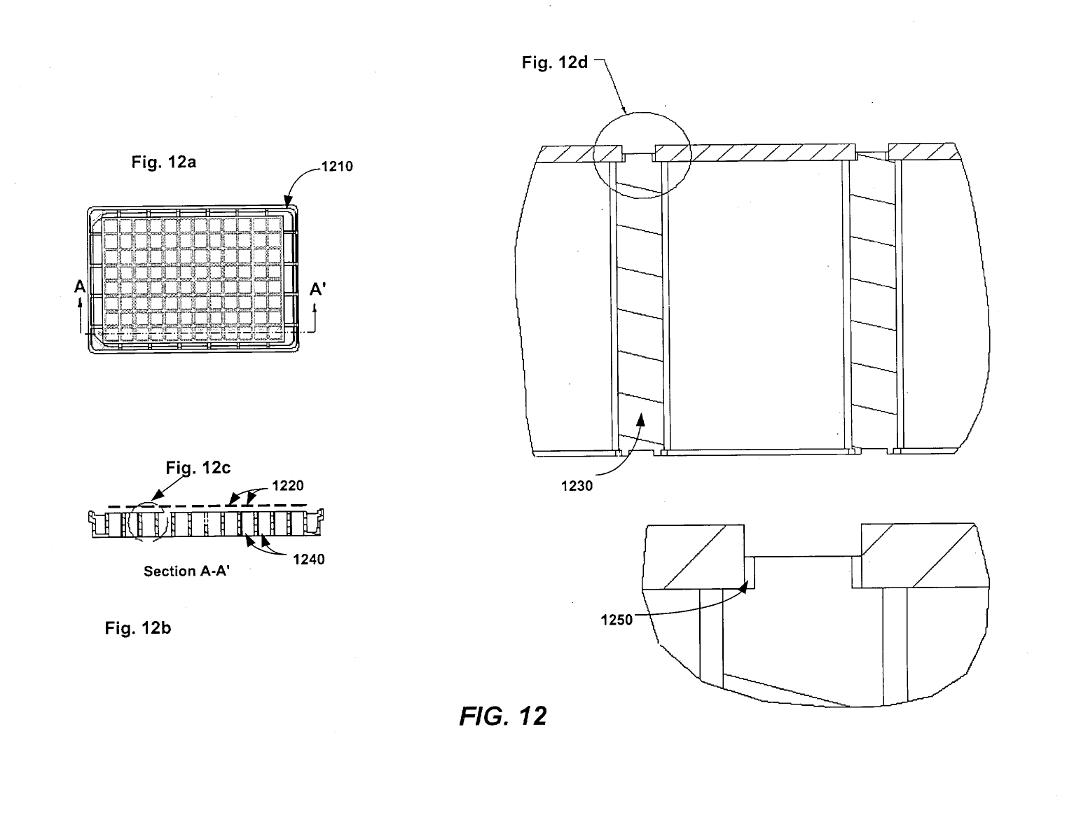

FIG. 13 depicts a further embodiment of this invention, wherein individual arrays are captured onto a grid by melting using a hot plate or ultrasonic horn.

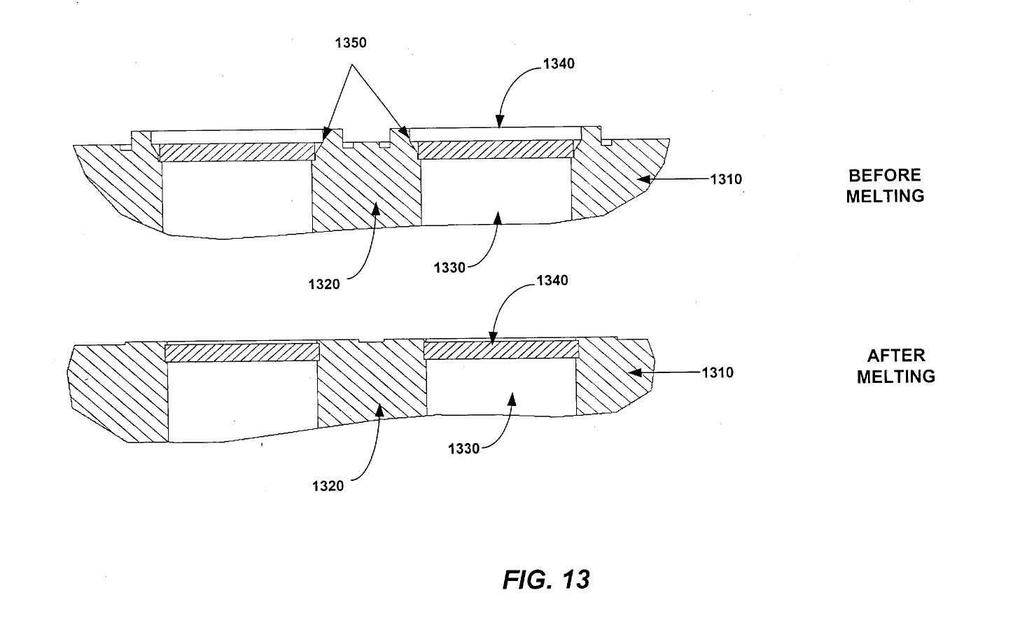

FIG. 14 depicts an embodiment of the invention, wherein a plurality of arrays from a diced wafer are transferred to a holding plate wherein the holding plate can be constructed of a single piece or a plurality of subplates which are a fraction of the plate.

FIG. 15 depicts a manifold making contact with a glue stamper to transfer adhesive to a well plate wherein the well plate and holding plate are aligned to transfer a plurality of arrays into the well plate.

FIG. 16 depicts an embodiment of the invention wherein an array plate is placed in a curing station.

### DETAILED DESCRIPTION OF THE INVENTION

Reference will now be made in detail to exemplary embodiments of the invention. While the invention will be described in conjunction with the exemplary embodiments, it will be understood that they are not intended to limit the invention to these embodiments. On the contrary, the invention is intended to cover alternatives, modifications and equivalents, which may be included within the spirit and scope of the invention.

The invention therefore relates to diverse fields impacted by the nature of molecular interaction, including chemistry, biology, medicine and diagnostics. The ability to do so would be advantageous in settings in which large amounts of information are required quickly, such as in clinical diagnostic laboratories or in large-scale undertakings such as the Human Genome Project.

The present invention has many preferred embodiments and relies on many patents, applications and other references for details known to those of the art. Therefore, when a patent, application, or other reference is cited or repeated below, it should be understood that it is incorporated by reference in its entirety for all purposes as well as for the proposition that is recited.

As used in this application, the singular form “a,” “an,” and “the” include plural references unless the context clearly dictates otherwise. For example, the term “an agent” includes a plurality of agents, including mixtures thereof.

An individual is not limited to a human being but may also be other organisms including but not limited to mammals, plants, bacteria, or cells derived from any of the above.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, New York, Gait, “Oligonucleotide Synthesis: A Practical Approach” 1984, IRL Press, London, Nelson and Cox (2000), Lehninger, Principles of Biochemistry 3rd Ed., W. H. Freeman Pub., New York, N.Y. and Berg et al. (2002) Biochemistry, 5th Ed., W. H. Freeman Pub., New York, N.Y., all of which are herein incorporated in their entirety by reference for all purposes.

The present invention can employ solid substrates, including arrays in some preferred embodiments. Methods and techniques applicable to polymer (including protein) array synthesis have been described in U.S. Ser. Nos. 09/536,841, WO 00/58516, U.S. Pat. Nos. 5,143,854, 5,242,974, 5,252,743, 5,324,633, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846 and 6,428,752, in PCT Applications Nos. PCT/US99/00730 (International Publication Number WO 99/36760) and PCT/US01/04285, which are all incorporated herein by reference in their entirety for all purposes.

Patents that describe synthesis techniques in specific embodiments include U.S. Pat. Nos. 5,412,087, 6,147,205, 6,262,216, 6,310,189, 5,889,165, and 5,959,098. Nucleic acid arrays are described in many of the above patents, but the same techniques are applied to polypeptide arrays.

Nucleic acid arrays that are useful in the present invention include those that are commercially available from Affymetrix (Santa Clara, Calif.) under the brand name GeneChip®. Example arrays are shown on the website at affymetrix.com.

The present invention also contemplates many uses for polymers attached to solid substrates. These uses include gene expression monitoring, profiling, library screening, genotyping and diagnostics. Gene expression monitoring, and profiling methods can be shown in U.S. Pat. Nos. 5,800,992, 6,013,449, 6,020,135, 6,033,860, 6,040,138, 6,177,248 and 6,309,822. Genotyping and uses therefore are shown in USSN 60/319,253, 10/013,598, and U.S. Pat. Nos. 5,856,092, 6,300,063, 5,858,659, 6,284,460, 6,361,947, 6,368,799 and 6,333,179. Other uses are embodied in U.S. Pat. Nos. 5,871,928, 5,902,723, 6,045,996, 5,541,061, and 6,197,506.

The present invention also contemplates sample preparation methods in certain preferred embodiments. Prior to or concurrent with genotyping, the genomic sample may be amplified by a variety of mechanisms, some of which may employ PCR. See, e.g., PCR Technology: Principles and Applications for DNA Amplification (Ed. H. A. Erlich, Freeman Press, N.Y., N.Y., 1992); PCR Protocols: A Guide to Methods and Applications (Eds. Innis, et al., Academic Press, San Diego, Calif., 1990); Mattila et al., Nucleic Acids Res. 19, 4967 (1991); Eckert et al., PCR Methods and Applications 1, 17 (1991); PCR (Eds. McPherson et al., IRL Press, Oxford); and U.S. Pat. Nos. 4,683,202, 4,683,195, 4,800,159 4,965,188, and 5,333,675, and each of which is incorporated herein by reference in their entireties for all purposes. The sample may be amplified on the array. See, for example, U.S. Pat. No. 6,300,070 and U.S. patent application No. 09/513,300, which are incorporated herein by reference.

Other suitable amplification methods include the ligase chain reaction (LCR) (e.g., Wu and Wallace, Genomics 4, 560 (1989), Landegren et al., Science 241, 1077 (1988) and Barringer et al. Gene 89:117 (1990)), transcription amplification (Kwoh et al., Proc. Natl. Acad. Sci. USA 86, 1173 (1989) and WO88/10315), self-sustained sequence replication (Guatelli et al., Proc. Nat. Acad. Sci. USA, 87, 1874 (1990) and WO90/06995), selective amplification of target polynucleotide sequences (U.S. Pat. No. 6,410,276), consensus sequence primed polymerase chain reaction (CP-PCR) (U.S. Pat. No. 4,437,975), arbitrarily primed polymerase chain reaction (AP-PCR) (U.S. Pat. Nos. 5,413,909, 5,861,245) and nucleic acid based sequence amplification (NABSA). (See, U.S. Pat. Nos. 5,409,818, 5,554,517, and 6,063,603, each of which is incorporated herein by reference). Other amplification methods that may be used are described in, U.S. Pat. Nos. 5,242,794, 5,494,810, 4,988,617 and in U.S. Ser. No. 09/854,317, each of which is incorporated herein by reference.

Additional methods of sample preparation and techniques for reducing the complexity of a nucleic sample are described in Dong et al., Genome Research 11, 1418 (2001), in U.S. Pat. Nos. 6,361,947, 6,391,592 and U.S. patent application Nos. 09/916,135, 09/920,491, 09/910,292, and 10/013,598.

Methods for conducting polynucleotide hybridization assays have been well developed in the art. Hybridization assay procedures and conditions will vary depending on the application and are selected in accordance with the general binding methods known including those referred to in: Maniatis et al. Molecular Cloning: A Laboratory Manual (2nd Ed. Cold Spring Harbor, N.Y, 1989); Berger and Kimmel Methods in Enzymology, Vol. 152, Guide to Molecular Cloning Techniques (Academic Press, Inc., San Diego, Calif., 1987); Young and Davism, P.N.A.S, 80: 1194 (1983). Methods and apparatus for carrying out repeated and controlled hybridization reactions have been described in U.S. Pat. Nos. 5,871,928, 5,874,219, 6,045,996 and 6,386,749, 6,391,623 each of which are incorporated herein by reference

The present invention also contemplates signal detection of hybridization between ligands in certain preferred embodirments. See U.S. Pat. Nos. 5,143,854, 5,578,832; 5,631,734; 5,834,758; 5,936,324; 5,981,956; 6,025,601; 6,141,096; 6,185,030; 6,201,639; 6,218,803; and 6,225,625, in U.S. patent application No. 60/364,731 and in PCT Application PCT/US99/06097 (published as WO99/47964), each of which also is hereby incorporated by reference in its entirety for all purposes.

Methods and apparatus for signal detection and processing of intensity data are disclosed in, for example, U.S. Pat. Nos. 5,143,854, 5,547,839, 5,578,832, 5,631,734, 5,800,992, 5,834,758; 5,856,092, 5,902,723, 5,936,324, 5,981,956, 6,025,601, 6,090,555, 6,141,096, 6,185,030, 6,201,639; 6,218,803; and 6,225,625, in U.S. patent application No. 60/364,731 and in PCT Application PCT/US99/06097 (published as WO99/47964), each of which also is hereby incorporated by reference in its entirety for all purposes.

The practice of the present invention may also employ conventional biology methods, software and systems. Computer software products of the invention typically include computer readable medium having computer-executable instructions for performing the logic steps of the method of the invention. Suitable computer readable medium include floppy disk, CD-ROM/DVD/DVD-ROM, hard-disk drive, flash memory, ROM/RAM, magnetic tapes and etc. The computer executable instructions may be written in a suitable computer language or combination of several languages. Basic computational biology methods are described in, e.g. Setubal and Meidanis et al., Introduction to Computational B iology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2nd ed., 2001). See U.S. Pat. No. 6,420,108.

The present invention may also make use of various computer program products and software for a variety of purposes, such as probe design, management of data, analysis, and instrument operation. See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170.

The present invention may also make use of the several embodiments of the array or arrays and the processing described in U.S. Pat. Nos. 5,545,531 and 5,874,219. These patents are incorporated herein by reference in their entireties for all purposes.

Additionally, the present invention may have preferred embodiments that include methods for providing genetic information over networks such as the Internet as shown in U.S. patent application Nos. 10/063,559, 60/349,546, 60/376,003, 60/394,574, 60/403,381.

Definitions

An “array” is an intentionally created collection of molecules which can be prepared either synthetically or biosynthetically. The molecules in the array can be identical or different from each other. The array can assume a variety of formats, e.g., libraries of soluble molecules; libraries of compounds tethered to resin beads, silica chips, or other solid supports.

Array Plate or a Plate a body having a plurality of arrays in which each array is separated from the other arrays by a physical barrier resistant to the passage of liquids and forming an area or space, referred to as a well.

Nucleic acid library or array is an intentionally created collection of nucleic acids which can be prepared either synthetically or biosynthetically and screened for biological activity in a variety of different formats (e.g., libraries of soluble molecules; and libraries of oligos tethered to resin beads, silica chips, or other solid supports). Additionally, the term “array” is meant to include those libraries of nucleic acids which can be prepared by spotting nucleic acids of essentially any length (e.g., from 1 to about 1000 nucleotide monomers in length) onto a substrate. The term “nucleic acid” as used herein refers to a polymeric form of nucleotides of any length, either ribonucleotides, deoxyribonucleotides or peptide nucleic acids (PNAs) as described in U.S. Pat. No. 6,156,501 that comprise purine and pyrimidine bases, or other natural, chemically or biochemically modified, non-natural, or derivatized nucleotide bases. The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. The sequence of nucleotides may be interrupted by non-nucleotide components. Thus the terms nucleoside, nucleotide, deoxynucleoside and deoxynucleotide generally include analogs such as those described herein. These analogs are those molecules having some structural features in common with a naturally occurring nucleoside or nucleotide such that when incorporated into a nucleic acid or oligonucleoside sequence, they allow hybridization with a naturally occurring nucleic acid sequence in solution. Typically, these analogs are derived from naturally occurring nucleosides and nucleotides by replacing and/or modifying the base, the ribose or the phosphodiester moiety. The changes can be tailor made to stabilize or destabilize hybrid formation or enhance the specificity of hybridization with a complementary nucleic acid sequence as desired.

Biopolymer or biological polymer: is intended to mean repeating units of biological or chemical moieties. Representative biopolymers include, but are not limited to, nucleic acids, oligonucleotides, amino acids, proteins, peptides, hormones, oligosaccharides, lipids, glycolipids, lipopolysaccharides, phospholipids, synthetic analogues of the foregoing, including, but not limited to, inverted nucleotides, peptide nucleic acids, Meta-DNA, and combinations of the above. “Biopolymer synthesis” is intended to encompass the synthetic production, both organic and inorganic, of a biopolymer.

Related to a bioploymer is a “biomonomer” which is intended to mean a single unit of biopolymer, or a single unit which is not part of a biopolymer. Thus, for example, a nucleotide is a biomonomer within an oligonucleotide biopolymer, and an amino acid is a biomonomer within a protein or peptide biopolymer; avidin, biotin, antibodies, antibody fragments, etc., for example, are also biomonomers.

Initiation Biomonomer: or “initiator biomonomer” is meant to indicate the first biomonomer which is covalently attached via reactive nucleophiles to the surface of the polymer, or the first biomonomer which is attached to a linker or spacer arm attached to the polymer, the linker or spacer arm being attached to the polymer via reactive nucleophiles.

Clamping plate: Refers to a device used for fastening two or more parts.

Complementary: Refers to the hybridization or base pairing between nucleotides or nucleic acids, such as, for instance, between the two strands of a double stranded DNA molecule or between an oligonucleotide primer and a primer binding site on a single stranded nucleic acid to be sequenced or amplified. Complementary nucleotides are, generally, A and T (or A and U), or C and G. Two single stranded RNA or DNA molecules are said to be substantially complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, substantial complementary exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See, M. Kanehisa Nucleic Acids Res. 12:203 (1984), incorporated herein by reference.

Combinatorial Synthesis Strategy: A combinatorial synthesis strategy is an ordered strategy for parallel synthesis of diverse polymer sequences by sequential addition of reagents which may be represented by a reactant matrix and a switch matrix, the product of which is a product matrix. A reactant matrix is a 1 column by m row matrix of the building blocks to be added. The switch matrix is all or a subset of the binary numbers, preferably ordered, between 1 and m arranged in columns. A “binary strategy” is one in which at least two successive steps illuminate a portion, often half, of a region of interest on the substrate. In a binary synthesis strategy, all possible compounds which can be formed from an ordered set of reactants are formed. In most preferred embodiments, binary synthesis refers to a synthesis strategy which also factors a previous addition step. For example, a strategy in which a switch matrix for a masking strategy halves regions that were previously illuminated, illuminating about half of the previously illuminated region and protecting the remaining half (while also protecting about half of previously protected regions and illuminating about half of previously protected regions). It will be recognized that binary rounds may be interspersed with non-binary rounds and that only a portion of a substrate may be subjected to a binary scheme. A combinatorial “masking” strategy is a synthesis which uses light or other spatially selective deprotecting or activating agents to remove protecting groups from materials for addition of other materials such as amino acids.

Effective amount refers to an amount sufficient to induce a desired result.

Excitation energy refers to energy used to energize a detectable label for detection, for example illuminating a fluorescent label. Devices for this use include coherent light or non coherent light, such as lasers, UV light, light emitting diodes, an incandescent light source, or any other light or other electromagnetic source of energy having a wavelength in the excitation band of an excitable label, or capable of providing detectable transmitted, reflective, or diffused radiation.

Gaskets or o-ring refers to any of a wide variety of seals or packings used between joined parts to prevent the escape of a gas or fluid. Gaskets or o-rings can be made of materials such as elastomer.

Genome is all the genetic material in the chromosomes of an organism. DNA derived from the genetic material in the chromosomes of a particular organism is genomic DNA. A genomic library is a collection of clones made from a set of randomly generated overlapping DNA fragments representing the entire genome of an organism.

Glue manifold: Adhesive dispensing device. A glue manifold is a specifically designed fluidic device that can distribute the adhesive through channels to a receptacle plate similar to the array plate format.

Glue Stamper: A plate which is used for controlled application of adhesive to an array plate. The glue stamper could be made of any suitable materials such as an elastomer.

Holding plate: A body for temporal placing of a set of arrays before they are connected to a well plate.

Hybridization conditions will typically include salt concentrations of less than about 1M, more usually less than about 500 mM and preferably less than about 200 mM. Hybridization temperatures can be as low as 5° C., but are typically greater than 22° C., more typically greater than about 30° C., and preferably in excess of about 37° C. Longer fragments may require higher hybridization temperatures for specific hybridization. As other factors may affect the stringency of hybridization, including base composition and length of the complementary strands, presence of organic solvents and extent of base mismatching, the combination of parameters is more important than the absolute measure of any one alone.

Hybridizations, e.g., allele-specific probe hybridizations, are generally performed under stringent conditions. For example, conditions where the salt concentration is no more than about 1 Molar (M) and a temperature of at least 25 degrees-Celcius (° C.), e.g., 750 mM NaCl, 50 mM NaPhosphate, 5 mM EDTA, pH 7.4 (5×SSPE)and a temperature of from about 25 to about 30° C.

Hybridizations are usually performed under stringent conditions, for example, at a salt concentration of no more than 1 M and a temperature of at least 25EC. For example, conditions of 5×SSPE (750 mM NaCl, 50 mM NaPhosphate, 5 mM EDTA, pH 7.4) and a temperature of 25-30° C. are suitable for allele-specific probe hybridizations. For stringent conditions, see, for example, Sambrook, Fritsche and Maniatis. “Molecular Cloning A laboratory Manual” 2nd Ed. Cold Spring Harbor Press (1989) which is hereby incorporated by reference in its entirety for all purposes above.

The term “hybridization” refers to the process in which two single-stranded polynucleotides bind non-covalently to form a stable double-stranded polynucleotide; triple-stranded hybridization is also theoretically possible. The resulting (usually) double-stranded polynucleotide is a “hybrid.” The proportion of the population of polynucleotides that forms stable hybrids is referred to herein as the “degree of hybridization.”

Hybridization probes are oligonucleotides capable of binding in a base-specific manner to a complementary strand of nucleic acid. Such probes include peptide nucleic acids, as described in Nielsen et al., Science 254, 1497-1500 (1991), and other nucleic acid analogs and nucleic acid mimetics. See U.S. Pat. No. 6,156,501.

Hybridizing specifically to: refers to the binding, duplexing, or hybridizing of a molecule substantially to or only to a particular nucleotide sequence or sequences under stringent conditions when that sequence is present in a complex mixture (e.g., total cellular) DNA or RNA.

Isolated nucleic acid is an object species invention that is the predominant species present (i.e., on a molar basis it is more abundant than any other individual species in the composition). Preferably, an isolated nucleic acid comprises at least about 50, 80 or 90% (on a molar basis) of all macromolecular species present. Most preferably, the object species is purified to essential homogeneity (contaminant species cannot be detected in the composition by conventional detection methods).

Label for example, a luminescent label, a light scattering label or a radioactive label. Fluorescent labels include, inter alia, the commercially available fluorescein phosphoramidites such as Fluoreprime (Pharmacia), Fluoredite (Millipore) and FAM (ABI). See U.S. Pat. No. 6,287,778.

Ligand: A ligand is a molecule that is recognized by a particular receptor. The agent bound by or reacting with a receptor is called a “ligand,” a term which is definitionally meaningful only in terms of its counterpart receptor. The term “ligand” does not imply any particular molecular size or other structural or compositional feature other than that the substance in question is capable of binding or otherwise interacting with the receptor. Also, a ligand may serve either as the natural ligand to which the receptor binds, or as a functional analogue that may act as an agonist or antagonist. Examples of ligands that can be investigated by this invention include, but are not restricted to, agonists and antagonists for cell membrane receptors, toxins and venoms, viral epitopes, hormones (e.g., opiates, steroids, etc.), hormone receptors, peptides, enzymes, enzyme substrates, substrate analogs, transition state analogs, cofactors, drugs, proteins, and antibodies.

Linkage disequilibrium or allelic association means the preferential association of a particular allele or genetic marker with a specific allele, or genetic marker at a nearby chromosomal location more frequently than expected by chance for any particular allele frequency in the population. For example, if locus X has alleles a and b, which occur equally frequently, and linked locus Y has alleles c and d, which occur equally frequently, one would expect the combination ac to occur with a frequency of 0.25. If ac occurs more frequently, then alleles a and c are in linkage disequilibrium. Linkage disequilibrium may result from natural selection of certain combination of alleles or because an allele has been introduced into a population too recently to have reached equilibrium with linked alleles.

Microtiter plates are arrays of discrete wells that come in standard formats (96, 384 and 1536 wells) which are used for examination of the physical, chemical or biological characteristics of a quantity of samples in parallel.

Mixed population or complex population: refers to any sample containing both desired and undesired nucleic acids. As a non-limiting example, a complex population of nucleic acids may be total genomic DNA, total genomic RNA or a combination thereof. Moreover, a complex population of nucleic acids may have been enriched for a given population but include other undesirable populations. For example, a complex population of nucleic acids may be a sample which has been enriched for desired messenger RNA (mRNA) sequences but still includes some undesired ribosomal RNA sequences (rRNA).

Monomer: refers to any member of the set of molecules that can be joined together to form an oligomer or polymer. The set of monomers useful in the present invention includes, but is not restricted to, for the example of (poly)peptide synthesis, the set of L-amino acids, D-amino acids, or synthetic amino acids. As used herein, “monomer” refers to any member of a basis set for synthesis of an oligomer. For example, dimers of L-17 amino acids form a basis set of 400 “monomers” for synthesis of polypeptides. Different basis sets of monomers may be used at successive steps in the synthesis of a polymer. The term “monomer” also refers to a chemical subunit that can be combined with a different chemical subunit to form a compound larger than either subunit alone.

mRNA or mRNA transcripts: as used herein, include, but not limited to pre-mRNA transcript(s), transcript processing intermediates, mature mRNA(s) ready for translation and transcripts of the gene or genes, or nucleic acids derived from the mRNA transcript(s). Transcript processing may include splicing, editing and degradation. As used herein, a nucleic acid derived from an mRNA transcript refers to a nucleic acid for whose synthesis the mRNA transcript or a subsequence thereof has ultimately served as a template. Thus, a cDNA reverse transcribed from an mRNA, an RNA transcribed from that cDNA, a DNA amplified from the cDNA, an RNA transcribed from the amplified DNA, etc., are all derived from the mRNA transcript and detection of such derived products is indicative of the presence and/or abundance of the original transcript in a sample. Thus, mRNA derived samples include, but are not limited to, mRNA transcripts of the gene or genes, cDNA reverse transcribed from the mRNA, cRNA transcribed from the cDNA, DNA amplified from the genes, RNA transcribed from amplified DNA, and the like.

Nucleic acid library or array is an intentionally created collection of nucleic acids which can be prepared either synthetically or biosynthetically and screened for biological activity in a variety of different formats (e.g., libraries of soluble molecules; and libraries of oligos tethered to resin beads, silica chips, or other solid supports). Additionally, the term “array” is meant to include those libraries of nucleic acids which can be prepared by spotting nucleic acids of essentially any length (e.g., from 1 to about 1000 nucleotide monomers in length) onto a substrate. The term “nucleic acid” as used herein refers to a polymeric form of nucleotides of any length, either ribonucleotides, deoxyribonucleotides or peptide nucleic acids (PNAs), that comprise purine and pyrimidine bases, or other natural, chemically or biochemically modified, non-natural, or derivatized nucleotide bases. The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. The sequence of nucleotides may be interrupted by non-nucleotide components. Thus the terms nucleoside, nucleotide, deoxynucleoside and deoxynucleotide generally include analogs such as those described herein. These analogs are those molecules having some structural features in common with a naturally occurring nucleoside or nucleotide such that when incorporated into a nucleic acid or oligonucleoside sequence, they allow hybridization with a naturally occurring nucleic acid sequence in solution. Typically, these analogs are derived from naturally occurring nucleosides and nucleotides by replacing and/or modifying the base, the ribose or the phosphodiester moiety. The changes can be tailor made to stabilize or destabilize hybrid formation or enhance the specificity of hybridization with a complementary nucleic acid sequence as desired.

Nucleic acids according to the present invention may include any polymer or oligomer of pyrimidine and purine bases, preferably cytosine, thymine, and uracil, and adenine and guanine, respectively. See Albert L. Lehninger, PRINCIPLES OF BIOCHEMISTRY, at 793-800 (Worth Pub. 1982). Indeed, the present invention contemplates any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally-occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

An “oligonucleotide” or “polynucleotide” is a nucleic acid ranging from at least 2, preferable at least 8, and more preferably at least 20 nucleotides in length or a compound that specifically hybridizes to a polynucleotide. Polynucleotides of the present invention include sequences of deoxyribonucleic acid (DNA) or ribonucleic acid (RNA) which may be isolated from natural sources, recombinantly produced or artificially synthesized and mimetics thereof. A further example of a polynucleotide of the present invention may be peptide nucleic acid (PNA). The invention also encompasses situations in which there is a nontraditional base pairing such as Hoogsteen base pairing which has been identified in certain tRNA molecules and postulated to exist in a triple helix. “Polynucleotide” and “oligonucleotide” are used interchangeably in this application.

Pick up Plate: A device to perform a transfer of arrays into an array plate for example with the use of suction.

Probe: A probe is a surface-immobilized molecule that can be recognized by a particular target. Examples of probes that can be investigated by this invention include, but are not restricted to, agonists and antagonists for cell membrane receptors, toxins and venoms, viral epitopes, hormones (e.g., opioid peptides, steroids, etc.), hormone receptors, peptides, enzymes, enzyme substrates, cofactors, drugs, lectins, sugars, oligonucleotides, nucleic acids, oligosaccharides, proteins, and monoclonal antibodies.

Primer is a single-stranded oligonucleotide capable of acting as a point of initiation for template-directed DNA synthesis under suitable conditions e.g., buffer and temperature, in the presence of four different nucleoside triphosphates and an agent for polymerization, such as, for example, DNA or RNA polymerase or reverse transcriptase. The length of the primer, in any given case, depends on, for example, the intended use of the primer, and generally ranges from 15 to 20, 25, 30 nucleotides. Short primer molecules generally require cooler temperatures to form sufficiently stable hybrid complexes with the template. A primer need not reflect the exact sequence of the template but must be sufficiently complementary to hybridize with such template. The primer site is the area of the template to which a primer hybridizes. The primer pair is a set of primers including a 5′ upstream primer that hybridizes with the 5′ end of the sequence to be amplified and a 3′ downstream primer that hybridizes with the complement of the 3′ end of the sequence to be amplified.

Polymorphism refers to the occurrence of two or more genetically determined alternative sequences or alleles in a population. A polymorphic marker or site is the locus at which divergence occurs. Preferred markers have at least two alleles, each occurring at frequency of greater than 1%, and more preferably greater than 10% or 20% of a selected population. A polymorphism may comprise one or more base changes, an insertion, a repeat, or a deletion. A polymorphic locus may be as small as one base pair. Polymorphic markers include restriction fragment length polymorphisms, variable number of tandem repeats (VNTR's), hypervariable regions, minisatellites, dinucleotide repeats, trinucleotide repeats, tetranucleotide repeats, simple sequence repeats, and insertion elements such as Alu. The first identified allelic form is arbitrarily designated as the reference form and other allelic forms are designated as alternative or variant alleles. The allelic form occurring most frequently in a selected population is sometimes referred to as the wildtype form. Diploid organisms may be homozygous or heterozygous for allelic forms. A diallelic polymorphism has two forms. A triallelic polymorphism has three forms. Single nucleotide polymorphisms (SNPs) are included in polymorphisms.

Reader or plate reader is a device which is used to identify hybridization events on an array, such as the hybridization between a nucleic acid probe on the array and a fluorescently labeled target. Readers are known in the art and are commercially available through Affymetrix, Santa Clara Calif. and other companies. Generally, they involve the use of an excitation energy (such as a laser) to illuminate a fluorescently labeled target nucleic acid that has hybridized to the probe. Then, the reemitted radiation (at a different wavelength than the excitation energy) is detected using devices such as a CCD, PMT, photodiode, or similar devices to register the collected emissions. See U.S. Pat. No. 6,225,625.

Receptor: A molecule that has an affinity for a given ligand. Receptors may be naturally-occurring or manmade molecules. Also, they can be employed in their unaltered state or as aggregates with other species. Receptors may be attached, covalently or noncovalently, to a binding member, either directly or via a specific binding substance. Examples of receptors which can be employed by this invention include, but are not restricted to, antibodies, cell membrane receptors, monoclonal antibodies and antisera reactive with specific antigenic determinants (such as on viruses, cells or other materials), drugs, polynucleotides, nucleic acids, peptides, cofactors, lectins, sugars, polysaccharides, cells, cellular membranes, and organelles. Receptors are sometimes referred to in the art as anti-ligands. As the term receptors is used herein, no difference in meaning is intended. A “Ligand Receptor Pair” is formed when two macromolecules have combined through molecular recognition to form a complex. Other examples of receptors which can be investigated by this invention include but are not restricted to those molecules shown in U.S. Pat. No. 5,143,854, which is hereby incorporated by reference in its entirety.

“Solid support”, “support”, and “substrate” are used interchangeably and refer to a material or group of materials having a rigid or semi-rigid surface or surfaces. In many embodiments, at least one surface of the solid support will be substantially flat, although in some embodiments it may be desirable to physically separate synthesis regions for different compounds with, for example, wells, raised regions, pins, etched trenches, or the like. According to other embodiments, the solid support(s) will take the form of beads, resins, gels, microspheres, or other geometric configurations. See U.S. Pat. No. 5,744,305 for exemplary substrates.

Subplate or subset array plate: Is a pre-assembled set of a fraction of a holding plate which will then be assembled into a 96-chip set format.

Target: A molecule that has an affinity for a given probe. Targets may be naturally-occurring or man-made molecules. Also, they can be employed in their unaltered state or as aggregates with other species. Targets may be attached, covalently or noncovalently, to a binding member, either directly or via a specific binding substance. Examples of targets which can be employed by this invention include, but are not restricted to, antibodies, cell membrane receptors, monoclonal antibodies and antisera reactive with specific antigenic determinants (such as on viruses, cells or other materials), drugs, oligonucleotides, nucleic acids, peptides, cofactors, lectins, sugars, polysaccharides, cells, cellular membranes, and organelles. Targets are sometimes referred to in the art as anti-probes. As the term targets is used herein, no difference in meaning is intended. A “Probe Target Pair” is formed when two macromolecules have combined through molecular recognition to form a complex.

Wafer: A substrate having surface to which a plurality of arrays are bound. In a preferred embodiment, the arrays are synthesized on the surface of the substrate to create multiple arrays that are physically separate. In one preferred embodiment of a wafer, the arrays are physically separated by a distance of at least about 0.1, 0.25, 0.5, 1 or 1.5 millimeters. The arrays that are on the wafer may be identical, each one may be different, or there may be some combination thereof. Particularly preferred wafers are about 8″×8″ and are made using the photolithographic process.

Well plate: A body with a plurality of cavities open at both wherein the cavities form an area or space referred to as a well wherein each well will hold an array.

### GENERAL

This invention provides automated methods for concurrently processing multiple array assays. (i.e. See U.S. Pat. No. 5,545,531). The methods of this invention allow many tests to be set up and processed together.

In some preferred methods of this invention, a plate is provided having a plurality of wells. Each well may include one or more arrays. Test samples, which may contain target molecules, are introduced into the wells. A fluid handling device exposes the wells to a chosen set of reaction conditions by, for example, adding or removing fluid from the wells, maintaining the liquid in the wells at predetermined temperatures, and agitating the wells as required, thereby performing the test. Then, an array reader interrogates the probe arrays in the wells, thereby obtaining the results of the tests. A computer having an appropriate program can further analyze the results from the tests.

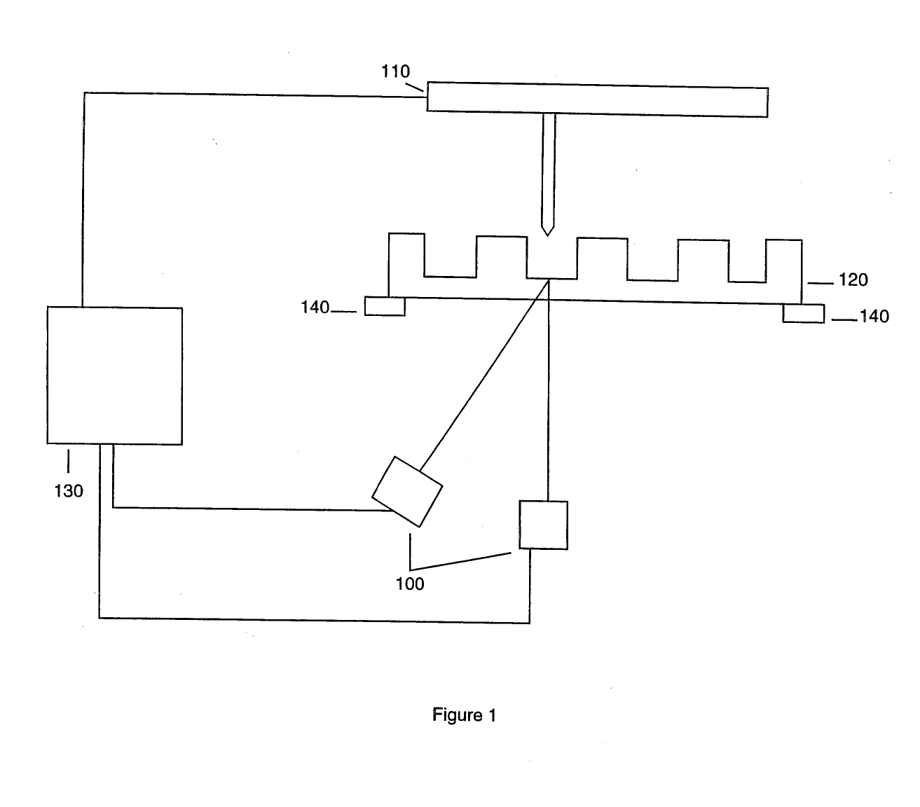

Referring to FIG. 1, one embodiment of the invention is a system for concurrently processing array assays. The system includes a plate reader 100, a fluid handling device 110, a plate 120 and, optionally, a computer 130. In operation, samples are placed in wells on the array plate 120 with fluid handling device 110. The plate optionally can be moved with a stage translation device 140. It should be understood that other devices may be employed to translate the plate relative to the reader. Reader 100 is used to identify where targets in the wells have bound to complementary probes. The system operates under control of computer 130 which may optionally interpret the results of the assay. See U.S. Pat. Nos. 6,225,625 and 5,835,758.

A. Reader

In assays performed on arrays, detectably labeled target molecules bind to probe molecules. Reading the results of an assay involves detecting a signal produced by the detectable label. Reading assays on an array plate requires a reader. Accordingly, locations at which target(s) bind with complementary probes can be identified by detecting the location of the label. Through knowledge of the characteristics/sequence of the probe versus location, characteristics of the target can be determined. The nature of the array reader depends upon the particular type of label attached to the target molecules. See U.S. Pat. Nos. 6,225,625; 6,040,138; 6,309,822; and 6,495,320.

The interaction between targets and probes can be characterized in terms of kinetics and thermodynamics. As such, it may be necessary to interrogate the array while in contact with a solution of labeled targets. In such systems, the detection system must be extremely selective, with the capacity to discriminate between surface-bound and solution-born targets. Also, in order to perform a quantitative analysis, the high-density of the probe sequences requires the system to have the capacity to distinguish between each feature site. The system also should have sensitivity to low signal and a large dynamic range.

In one embodiment, the reader includes a confocal detection device having a monochromatic or polychromatic light source, a focusing system for directing an excitation light from the light source to the substrate, a temperature controller for controlling the substrate temperature during a reaction, and a detector for detecting fluorescence emitted by the targets in response to the excitation light. The detector for detecting the fluorescent emissions from the substrate, in some embodiments, includes a photomultiplier tube. The location to which light is directed may be controlled by, for example, an x-y-z translation table. Translation of the x-y-z table, temperature control, and data collection are managed and recorded by an appropriately programmed digital computer.

Further details for methods of detecting fluorescently labeled materials on arrays are provided in U.S. Pat. Nos. 5,631,734; 6,225,625; and 5,835,758 incorporated herein by reference in their entirety for all purposes.

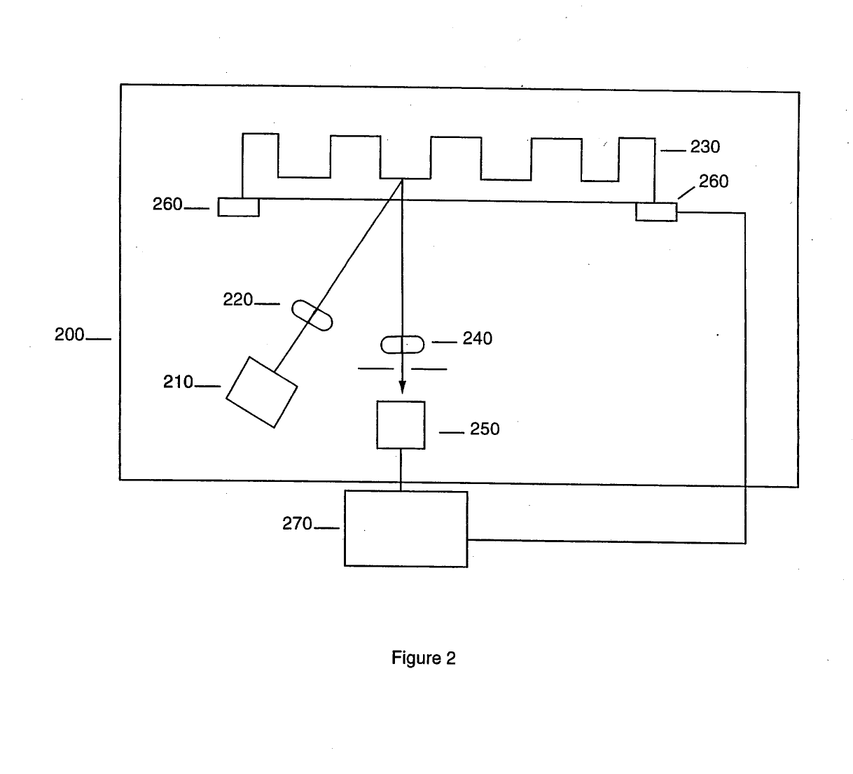

FIG. 2 illustrates a reader according to one specific embodiment. The reader comprises a body 200 for immobilizing the array plate. Excitation radiation, from an excitation source 210 having a first wavelength, passes through excitation optics 220 from preferably below the array. However, other directions may be employed. The light passes through the array plate since it is transparent to at least this wavelength of light. The excitation radiation excites a region of a probe array on the array plate 230. In response, labeled material on the sample emits radiation which has a wavelength that is different from the excitation wavelength. Collection optics 240, preferably also below the array (but may be located in another position), then collect the emission from the sample and image it onto a detector 250, which can house a CCD array, as described below. Alternate photo-detection devices can be used. The detector generates a signal proportional to the amount of radiation sensed thereon. The signals can be assembled to represent an image associated with the plurality of regions from which the emission originated.

According to one embodiment, a multi-axis translation stage 260 moves the array plate to position different wells to be scanned, and to allow different probe portions of a probe array to be interrogated. As a result, a 2-dimensional image of the probe arrays in each well is obtained. It should also be understood that relative translation between the detector, optics or excitation energy source, for example is possible.

The reader can include auto-focusing feature to maintain the sample in the focal plane of the excitation light throughout the scanning process. Further, a temperature controller may be employed to maintain the sample at a specific temperature while it is being scanned. The multi-axis translation stage, temperature controller, auto-focusing feature, and electronics associated with imaging and data collection are managed by an appropriately programmed digital computer 270.

In one embodiment, a beam is focused onto a spot of about 2 μm in diameter on the surface of the plate using, for example, the objective lens of a microscope or other optical means to control beam diameter. (See, e.g., U.S. Pat. No. 5,631,734.)

In another embodiment, fluorescent probes are employed in combination with CCD imaging systems. Details of this method are described in U.S. Pat. No. 5,578,832, incorporated herein by reference in its entirely. In many commercially available microplate readers, typically the light source is placed above a well, and a photodiode detector is below the well. In the present invention, the light source can be replaced with a higher power lamp or laser. In one embodiment, the standard absorption geometry is used, but the photodiode detector is replaced with a CCD camera and imaging optics to allow rapid imaging of the well. A series of Raman holographic or notch filters can be used in the optical path to eliminate the excitation light while allowing the emission to pass to the detector. In a variation of this method, a fiber optic imaging bundle is utilized to bring the light to the CCD detector. In another embodiment, the laser is placed below the array plate and light directed through the transparent wafer or base that forms the bottom of the array plate. In another embodiment, the CCD array is built into the wafer of the array plate.

The choice of the CCD array will depend on the number of probes in each array. If 2500 probes nominally arranged in a square (50×50) are examined, and 6 lines in each feature are sampled to obtain a good image, then a CCD array of 300×300 pixels is desirable in this area. However, if an individual well has 48,400 probes (220×220) then a CCD array with 1320×1320 pixels is desirable. CCD detectors are commercially available from, e.g., Princeton Instruments, which can meet either of these requirements.

In another embodiment, the detection device comprises a line scanner, as described in U.S. Pat. No. 5,578,832, incorporated herein by reference. Excitation optics focuses excitation light to a line at a sample, simultaneously scanning or imaging a strip of the sample. Surface bound labeled targets from the sample fluoresce in response to the light. Collection optics image the emission onto a linear array of light detectors. By employing confocal techniques, substantially only emission from the light's focal plane is imaged. Once a strip has been scanned, the data representing the 1-dimensional image are stored in the memory of a computer. According to one embodiment, a multi-axis translation stage moves the device at a constant velocity to continuously integrate and process data. Alternatively, galvometric scanners or rotating polyhedral mirrors may be employed to scan the excitation light across the sample. As a result, a 2-dimensional image of the sample is obtained. See U.S. Pat. Nos. 5,545,901, 6,207,960, and 6,335,824.

In another embodiment, collection optics direct the emission to a spectrograph which images an emission spectrum onto a 2-dimensional array of light detectors. By using a spectrograph, a full spectrally resolved image of the sample is obtained.

The read time for a full microtiter plate will depend on the photophysics of the fluorophore (i.e. fluorescence quantum yield and photodestruction yield) as well as the sensitivity of the detector. For fluorescein, sufficient signal-to-noise to read a chip image with a CCD detector can be obtained in about 30 seconds using 3 mW/cm² and 488 nm excitation from an Ar ion laser or lamp. By increasing the laser power, and switching to dyes such as CY3 or CY5 which have lower photodestruction yields and whose emission more closely matches the sensitivity maximum of the CCD detector, one easily is able to read each well in less than 5 seconds. Thus, an entire plate could be examined quantitatively in less than 10 minutes, even if the whole plate has over 4.5 million probes.

A computer can transform the data into another format for presentation. Data analysis can include the steps of determining, e.g., fluorescent intensity as a function of substrate position from the data collected, removing “outliers” (data deviating from a predetermined statistical distribution), and calculating the relative binding affinity of the targets from the remaining data. The resulting data can be displayed as an image with color in each region varying according to the light emission or binding affinity between targets and probes therein.

One application of this system when coupled with the CCD imaging system that speeds performance of the tests is to obtain results of the assay by examining the on- or off-rates of the hybridization. In one embodiment of this method, the amount of binding at each address is determined at several time points after the probes are contacted with the sample. The amount of total hybridization can be determined as a function of the kinetics of binding based on the amount of binding at each time point. Thus, it is not necessary to wait for equilibrium to be reached. The dependence of the hybridization rate for different oligonucleotides on temperature, sample agitation, washing conditions (e.g. pH, solvent characteristics, temperature) can easily be determined in order to maximize the conditions for rate and signal-to-noise. Alternative methods are described in Fodor et al., U.S. Pat. No. 5,324,633, incorporated herein by reference in its entirety for all purposes. See U.S. Pat. Nos. 6,040,138 and 6,495,320.

B. Fluid Handling Instruments and Assay Automation

Arrays generally include contacting an array with a sample under the selected reaction conditions, optionally washing the well to remove unreacted molecules, and analyzing the array for evidence of reaction between target molecules the probes. See U.S. Pat. No. 6,495,320. These steps involve handling fluids. The methods of this invention automate these steps so as to allow multiple assays to be performed concurrently. Accordingly, this invention employs automated fluid handling systems for concurrently performing the assay steps in each of the wells. Fluid handling allows uniform treatment of samples in the wells. Microtiter robotic and fluid-handling devices are available commercially, for example, from Tecan AG.

The plate is introduced into a holder in the fluid-handling device. This robotic device is programmed to set appropriate reaction conditions, such as temperature, add samples to the wells, incubate the test samples for an appropriate time, remove unreacted samples, wash the wells, add substrates as appropriate and perform detection assays. The particulars of the reaction conditions depends upon the purpose of the assay. For example, in a sequencing assay involving DNA hybridization, standard hybridization conditions are chosen. However, the assay may involve testing whether a sample contains target molecules that react to a probe under a specified set of reaction conditions. In this case, the reaction conditions are chosen accordingly.

C. Array Plates

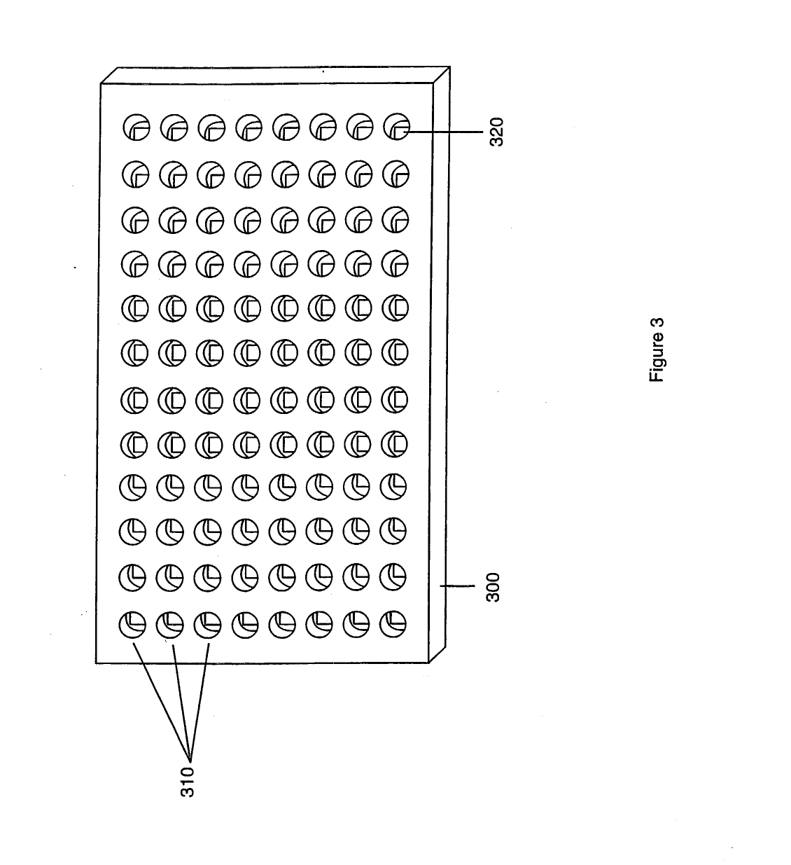

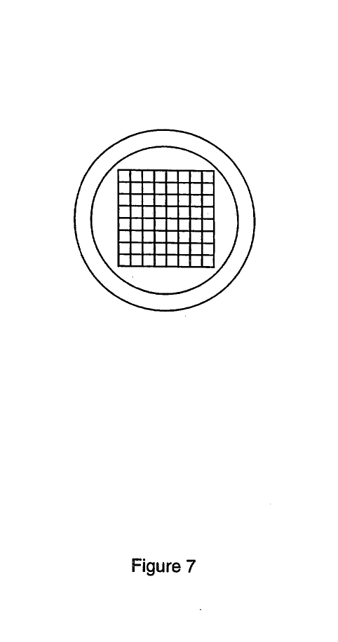

FIG. 3 depicts an example of an array plate 300 used in the methods of this invention based on the standard 96-well microtiter plate in which the arrays are located at the bottom of the wells. Array plates include a plurality of wells 310, each well defining an area or space for the introduction of a sample, and each well comprising an array 320, i.e., a substrate and a surface to which an array of probes is attached, the probes being exposed to the interior of the cavity. FIG. 7 shows a top-down view of a well of an array plate of this invention containing an array on the bottom surface of the well.

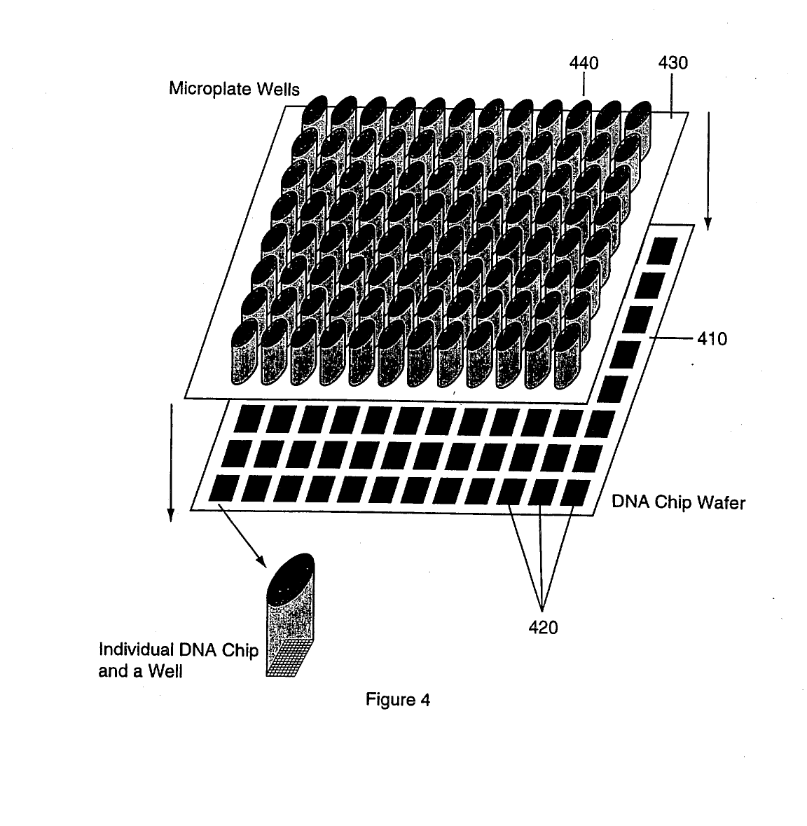

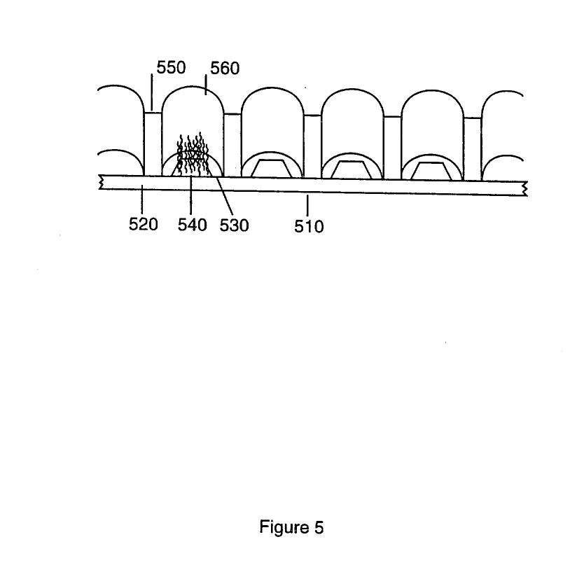

This invention contemplates a number of embodiments of the array plate. In a preferred embodiment, depicted in FIG. 4, the array plate includes two parts. One part is a wafer 410 that includes a plurality of arrays 420. The other part is the body 430 of the array plate that contains channels 440 that forn the well, but that are open at both ends until the array plate is attached. The body is attached to the surface of the wafer so as to close one end of the channels, thereby creating wells. The walls of the channels are placed on the wafer so that each surrounds and encloses an array. FIG. 5 depicts a cross-section of this embodiment, showing the wafer 510 having a substrate 520 (preferably transparent to light) and a surface 530 to which is attached an array of probes 540. A cavity encloses an array on the wafer, thereby creating wells 560. The wafer can be attached to the body by any attachment means known in the art, for example, gluing (e.g., by ultraviolet-curing adhesive or various sticking tapes), acoustic welding, sealing such as vacuum or suction sealing, or even by relying on the weight of the body on the wafer to resist the flow of fluids between wells.

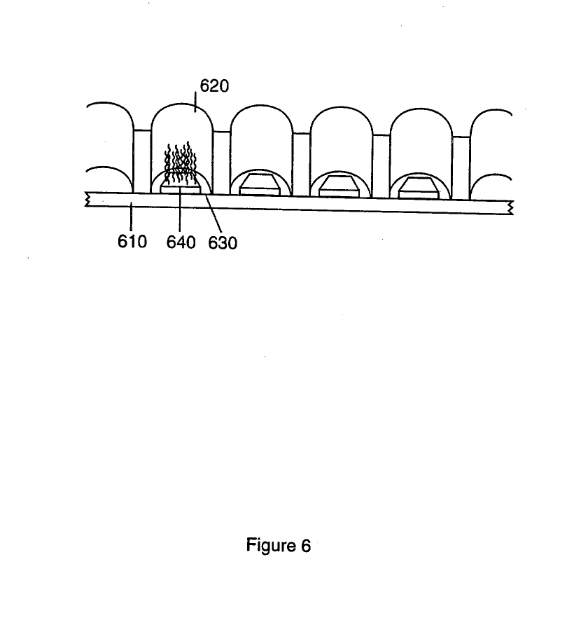

In another preferred embodiment, depicted in cross section in FIG. 6, the array plates include a body 610 having pre-formed wells 620, usually flat-bottomed. Individual arrays 630 are attached to the bottom of the wells so that the surface containing the array of probes 640 is exposed to the well space where the sample is to be placed.

In another embodiment, the array plate has a wafer having a plurality of probe arrays and a material resistant to the flow of a liquid sample that surrounds each probe array. For example, in an embodiment useful for testing aqueous-based samples, the wafer can be scored with waxes, tapes or other hydrophobic materials in the spaces between the arrays, forming cells that act as wells. The cells thus contain liquid applied to an array by resisting spillage over the barrier and into another cell. If the sample contains a non-aqueous solvent, such as an alcohol, the material is selected to be resistant to corrosion by the solvent.

The array plates of this invention have a plurality of wells that can be arrayed in a variety of ways. In one embodiment, the array plates have the general size and shape of standard-sized microtiter plates having 96 wells arranged in an 8×12 format. One advantage of this format is that instrumentation already exists for handling and reading assays on microtiter plates. Therefore, using such plates in array assays does not involve extensive re-engineering of commercially available fluid handling devices. However, the array plates can have other formats as well.

The material from which the body of the array plate is made depends upon the use to which it is to be put. In particular, this invention contemplates a variety of polymers already used for microtiter plates including, for example, (poly)tetrafluoroethylene (PTFE), (poly)vinylidenedifluoride (PVD), polypropylene, polystyrene, acrylonitrile butadiene-strene (ABS), Cyrolite G-20 Hi Flow (Acrylic Based Multipolymer Compound), or combinations thereof which are commercially available. When the assay is to be performed by sending an excitation beam through the bottom of the array plate collecting data through the bottom of the array plate, the body of the array plate and the substrate of the array should be transparent to the wavelengths of light being used.

The arrangement of probe arrays in the wells of a microplate depends on the particular application contemplated. For example, for diagnostic uses involving performing the same test on many samples, every well can have the same array of probes. If several different tests are to be performed on each sample, each row of the array plate can have the same array of probes and each column can contain a different array. Samples from a single patient are introduced into the wells of a particular column. Samples from a different patient are introduced into the wells of a different column. In still another embodiment, multiple patient samples are introduced into a single well. If a well indicates a “positive” result for a particular characteristic, the samples from each patient are then rerun, each in a different well, to determine which patient sample gave a positive result.

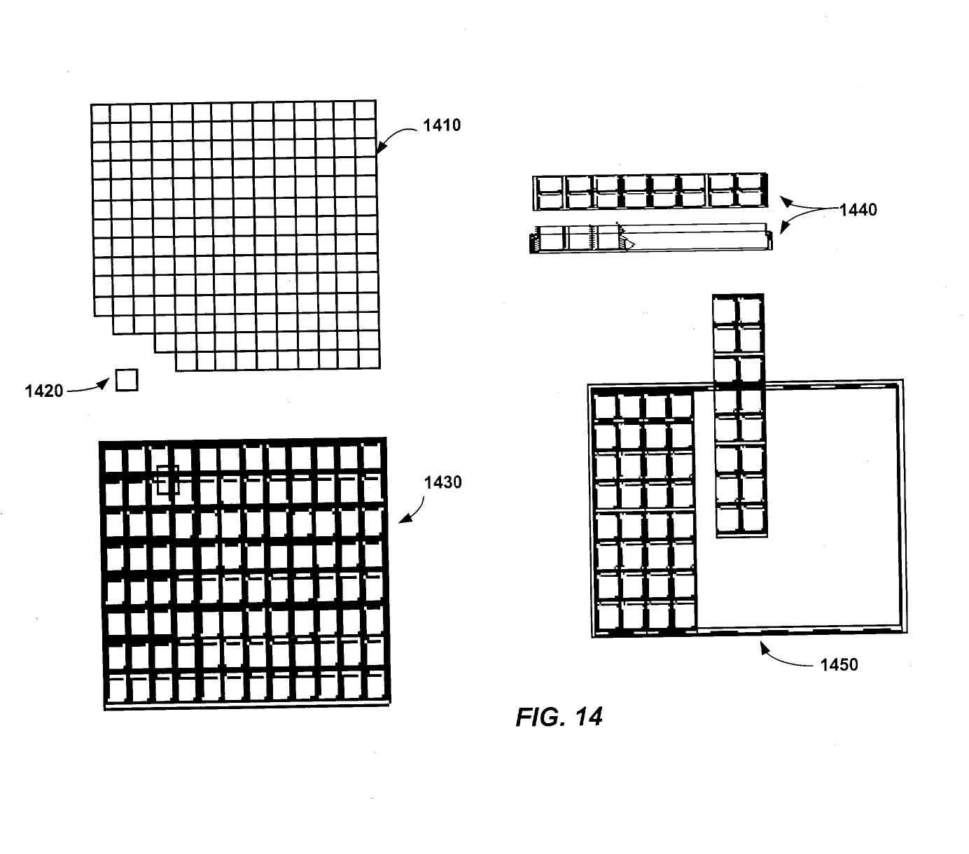

In one preferred embodiment of the invention depicted in FIG. 14, the process of manufacturing an array plate comprised of a plurality of arrays 1420 which can be selected from a plurality of diced wafers 1410 of different array products. The arrays 1420 from the diced wafers 1410 are selected and transferred individually to a holding plate 1430. This is a process commonly implemented in the semiconductor industry. Typically a pick and place instrument performance can exceed 2000-3000 dies per hour depending on the die size. The ability to take individual arrays 1420 from a diced wafer 1410 allows the potential for transfer of different types of array products from multiple sets of diced wafers, thus constructing an array plate of several different array products.

In one embodiment of the invention, the holding plate 1430 can be made of a single plate in a 96-array format or may be a subset array plate 1440 which is preassembled to be any fraction of a 96 array plate format 1450. Therefore adding flexibility and packaging capability. Each subset array plate 1430 can be manufactured in large volumes and separated for individual pounching and labeling. The end user can then take any combination of subset array plates and reassemble them into a 96 format for assay use on a high throughput instrument. Given the flexibility of assembly, the manufacturer can also pre-assemble these into a custom ordered 96-array set. The individual subset array sections can be molded with thermoplastic materials with snap-in features for assembly.

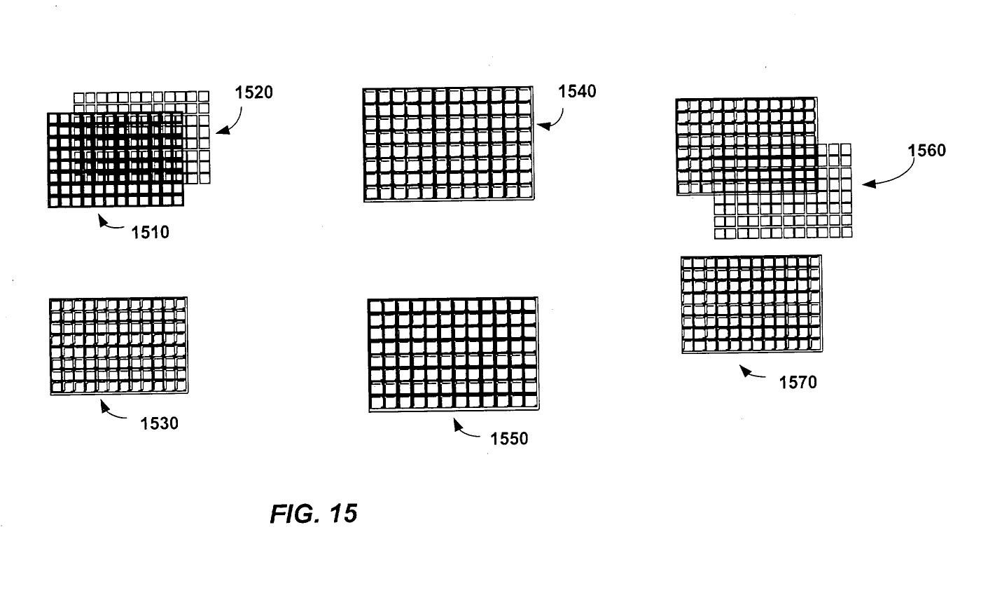

Referring to FIG. 15, in one embodiment of the invention, after the arrays are selected and transferred to a holding plate 1430, a glue manifold 1510 can dispense adhesive to a stamping pool. A glue stamper 1520 is then put in contact with the glue manifold 1510 and/or stamping pool. The glue stamper 1520 is then used to transfer adhesive to a well plate 1530. Stamping techniques like this are commonly used in labeling and silk screening to transfer ink to the product surfaces. Using a glue manifold 1510 to dispense adhesive to a stamping pool provides the advantage of controlling and ensuring dispense separately from the transfer and application of the adhesive to the well plate 1530 or any assembled subset of arrays 1440.

Once the well plate or assembled subset of arrays is prepared with adhesive 1540 and the holding plate has been filled with arrays 1550, then the well plate and the holding plate are aligned together 1560. A pick up plate is then used to transfer the arrays into the plate so as to create an array plate 1570. This pick up plate is designed to pick up all the arrays at the same time and accurately place them on the well plate or a subset array assembly as to form an array plate. The pick up plate also provides the seating pressure required to ensure that the arrays are installed properly for bonding.

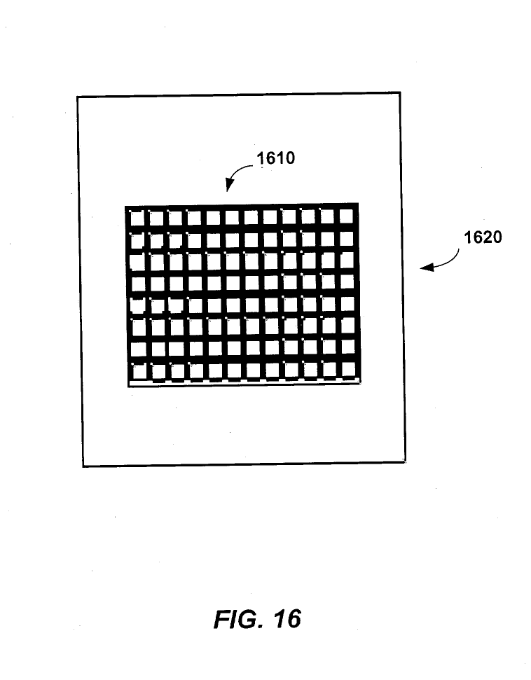

As depicted in FIG. 16, in one embodiment of the invention, after the arrays have been transferred into the well plate or subset array assembly, the array plate 1610 is then placed into a UV light station 1620 for curing of the adhesive. Since this process can be scaled to any volume desired, it is possible to place several array plates to the UV light station at the same time thus further shortening the total manufacturing time.

D. Arrays

The array plates used in the methods of this invention include arrays. The array of probe sequences can be fabricated on the array according to the pioneering techniques disclosed in U.S. Pat. Nos. 5,143,854, 5,744,305, 5,968,740, or 5,571,639, incorporated herein by reference for all purposes. The combination of photolithographic and fabrication techniques may, for example, enable each probe sequence (“feature”) to occupy a very small area (“site” or “location”) on the support. In some embodiments, this feature site may be as small as a few microns or even a single molecule. For example, a probe array of 0.25 mm² (about the size that would fit in a well of a typical 96-well microtiter plate) could have at least 10, 100, 1000, 10⁴, 10⁵ or 10⁶ features. In an alternative embodiment, such synthesis is performed according to the mechanical techniques disclosed in U.S. Pat. No. 5,384,261, incorporated herein by reference. Arrays can be made using alternative techniques including spotting. See U.S. Pat. No. 6,040,193.

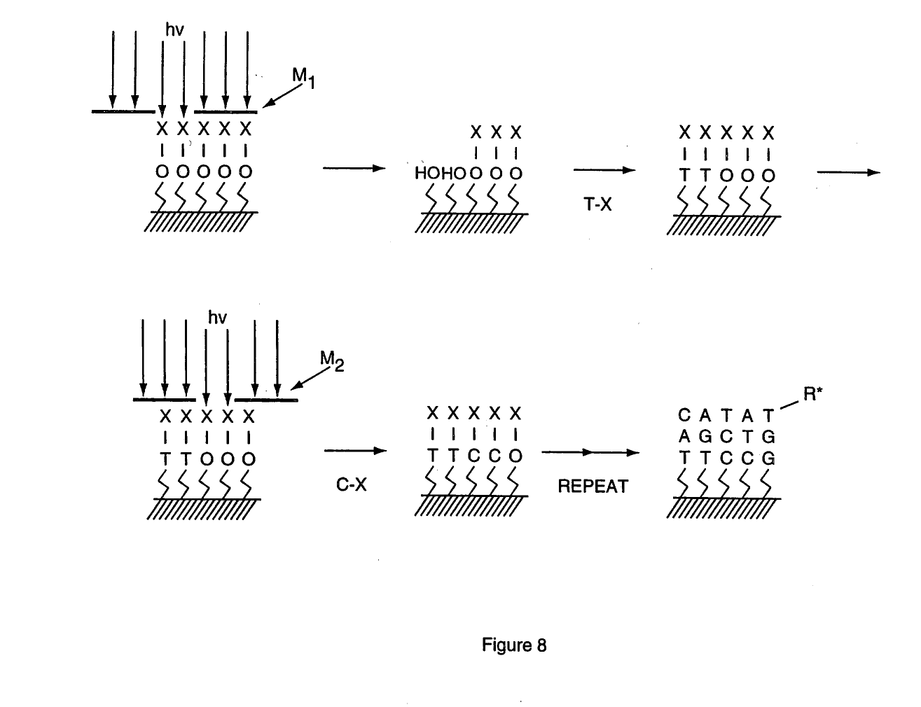

Referring to FIG. 8, in general, linker molecules, ˜O-X, are provided on a substrate. The substrate is preferably flat but may take on a variety of alternative surface configurations. For example, the substrate may contain raised or depressed regions on which the probes are located. The substrate and its surface preferably form a rigid support on which the sample can be formed. The substrate and its surface are also chosen to provide appropriate light-absorbing characteristics. For instance, the substrate may be functionalized glass, Si, Ge, GaAs, GaP, SiO₂, SiN₄, modified silicon, or any one of a wide variety of gels or polymers such as (poly)tetrafluoroethylene, (poly)vinylidenedifluoride, polystyrene, olycarbonate, polypropylene, or combinations thereof. Other substrate materials will be readily apparent to those of skill in the art upon review of this disclosure. In a preferred embodiment the substrate is flat glass or silica.

Surfaces on the solid substrate usually, though not always, are composed of the same material as the substrate. Thus, the surface may be composed of any of a wide variety of materials, for example, polymers, plastics, resins, polysaccharides, silica or silica-based materials, carbon, metals, inorganic glasses, membranes, or any of the above-listed substrate materials. In one embodiment, the surface will be optically transparent and will have surface Si—OH functionalities, such as those found on silica surfaces.

A terminal end of the linker molecules is provided with a reactive functional group protected with a photoremovable protective group, 0-X. Using lithographic methods, the photoremovable protective group is exposed to light, hv, through a mask, M₁, that exposes a selected portion of the surface, and removed from the linker molecules in first selected regions. The substrate is then washed or otherwise contacted with a first monomer that reacts with exposed functional groups on the linker molecules (˜T-X). In the case of nucleic acids, the monomer can be a phosphoramidite activated nucleoside protected at the 5′-hydroxyl with a photolabile protecting group. See U.S. Pat. No. 5,959,098.

A second set of selected regions, thereafter, exposed to light through a mask, M₂, and photoremovable protective group on the linker molecule/protected amino acid or nucleotide is removed at the second set of regions. The substrate is then contacted with a second monomer containing a photoremovable protective group for reaction with exposed functional groups. This process is repeated to selectively apply monomers until polymers of a desired length and desired chemical sequence are obtained. Photolabile groups are then optionally removed and the sequence is, thereafter, optionally capped. Side chain protective groups, if present, are also removed.

The general process of synthesizing probes by removing protective groups by exposure to light, coupling monomer units to the exposed active sites, and capping unreacted sites is referred to herein as “light-directed probe synthesis.” If the probe is an oligonucleotide, the process is referred to as “light-directed oligonucleotide synthesis” and so forth.

The probes can be made of any molecules whose synthesis involves sequential addition of units. This includes polymers composed of a series of attached units and molecules bearing a common skeleton to which various functional groups are added. Polymers useful as probes in this invention include, for example, both linear and cyclic polymers of nucleic acids, polysaccharides, phospholipids, and peptides having either α-, β-, or ω-amino acids, heteropolymers in which a known drug is covalently bound to any of the above, polyurethanes, polyesters, polycarbonates, polyureas, polyamides, polyethyleneimines, polyarylene sulfides, polysiloxanes, polyimides, polyacetates, or other polymers which will be apparent upon review of this disclosure. Molecules bearing a common skeleton include benzodiazepines and other small molecules, such as described in U.S. Pat. No. 5,288,514, incorporated herein by reference.

Preferably, probes are arrayed on an array in addressable rows and columns in which the dimensions of the array conform to the dimension of the array plate well. Technologies already have been developed to read information from such arrays. The amount of information that can be stored on each array plate depends on the lithographic density which is used to synthesize the wafer. For example, if each feature size is about 100 microns on a side, each array can have about 10,000 probe addresses in a 1 cm² area. An array plate having 96 wells would contain about 192,000 probes. However, if the arrays have a feature size of 20 microns on a side, each array can have close to 50,000 probes and the array plate would have over 4,800,000 probes.

The selection of probes and their organization in an array depends upon the use to which the array will be put. In one embodiment, the arrays are used to sequence or re-sequence nucleic acid molecules, or compare their sequence to a referent molecule. Re-sequencing nucleic acid molecules involves determining whether a particular molecule has any deviations from the sequence of reference molecule. For example, in one embodiment, the array plates are used to identify in a particular type of HIV in a set of patient samples. Tiling strategies for sequence checking of nucleic acids are described in International Patent Publication No. WO9511995, incorporated herein by reference.

In typical diagnostic applications, a solution containing one or more targets to be identified (i.e., samples from patients) contacts the probe array. The targets will bind or hybridize with complementary probe sequences. Accordingly, the probes will be selected to have sequences directed to (i.e., having at least some complementarity with) the target sequences to be detected, e.g., human or pathogen sequences. Generally, the targets are tagged with a detectable label. The detectable label can be, for example, a luminescent label, a light scattering label or a radioactive label. Accordingly, locations at which targets hybridize with complimentary probes can be identified by locating the markers. Based on the locations where hybridization occurs, information regarding the target sequences can be extracted. The existence of a mutation may be determined by comparing the target sequence with the wild type. See U.S. Pat. No. 6,309,822.

In a preferred embodiment, the detectable label is a luminescent label. Useful luminescent labels include fluorescent labels, chemi-luminescent labels, bio-luminescent labels, and colorimetric labels, among others. Most preferably, the label is a fluorescent label such as fluorescein, rhodamine, digoxigenin, texas red, cyanine-3, cyanine-5 and so forth. Fluorescent labels include, inter alia, the commercially available fluorescein phosphoramidites such as Fluoreprime (Pharmacia), Fluoredite (Millipore) and FAM (ABI). For example, the entire surface of the substrate is exposed to the activated fluorescent phosphoramidite, which reacts with all of the deprotected 5′-hydroxyl groups. Then the entire substrate is exposed to an alkaline solution (eg., 50% ethylenediamine in ethanol for 1-2 hours at room temperature). This is necessary to remove the protecting groups from the fluorescein tag.

To avoid self-quenching interactions between fluorophores on the surface of an array, the fluorescent tag monomer should be diluted with a non-fluorescent analog of equivalent reactivity. For example, in the case of the fluorescein phosphoramidites noted above, a 1:20 dilution of the reagent with a non-fluorescent phosphoramidite such as the standard 5′-DMT-nucleoside phosphoramidites, has been found to be suitable. Correction for background non-specific binding of the fluorescent reagent and other such effects can be determined by routine testing.

Useful light scattering labels include large colloids, and especially the metal colloids such as those from gold, selenium and titanium oxide. See U.S. Pat. No. 6,294,327.

Radioactive labels include, for example, ³²P. This label can be detected by a phosphoimager. Detection of course, depends on the resolution of the imager. Phosophoimagers are available having resolution of 50 microns. Accordingly, this label is currently useful with arrays having features of that size.

E. Uses

The methods of this invention will find particular use wherever high through-put of samples is required. In particular, this invention is useful in clinical settings and for sequencing large quantities of DNA, for example in connection with the Human Genome project.

The clinical setting requires performing the same test on many patient samples. The automated methods of this invention lend themselves to these uses when the test is one appropriately performed on an array. For example, a DNA array can determine the particular strain of a pathogenic organism based on characteristic DNA sequences of the strain. The advanced techniques based on these assays now can be introduced into the clinic. Fluid samples from several patients are introduced into the wells of an array plate and the assays are performed concurrently.

In some embodiments, it may be desirable to perform multiple tests on multiple patient samples concurrently. According to such embodiments, rows (or columns) of the microtiter plate will contain probe arrays for diagnosis of a particular disease or trait. For example, one row might contain probe arrays designed for a particular cancer, while other rows contain probe arrays for another cancer. Patient samples are then introduced into respective columns (or rows) of the microtiter plate. For example, one column may be used to introduce samples from patient “one,” another column for patient “two” etc. Accordingly, multiple diagnostic tests may be performed on multiple patients in parallel. In still further embodiments, multiple patient samples are introduced into a single well. In a particular well indicator the presence of a genetic disease or other characteristic, each patient sample is then individually processed to identify which patient exhibits that disease or trait. For relatively rarely occurring characteristics, further order-of-magnitude efficiency may be obtained according to this embodiment.

Particular assays that will find use in automation include those designed specifically to detect or identify particular variants of a pathogenic organism, such as HIV. Assays to detect or identify a human or animal gene are also contemplated. In one embodiment, the assay is the detection of a human gene variant that indicates existence of or predisposition to a genetic disease, either from acquired or inherited mutations in an individual DNA.

These include genetic diseases such as cystic fibrosis, diabetes, and muscular dystrophy, as well as diseases such as cancer (the P53 gene is relevant to some cancers), as disclosed in Patent Publication No. WO9511995, already incorporated by reference.

The present invention provides a substantially novel method for performing assays on arrays. While specific examples have been provided, the above description is illustrative and not restrictive. Many variations of the invention will become apparent to those of skill in the art upon review of this specification. The scope of the invention should, therefore, be determined not with reference to the above description, but instead should be determined with reference to the appended claims along with their full scope of equivalents.

All publications and patent documents cited in this application are incorporated by reference in their entirety for all purposes to the same extent as if each individual publication or patent document were so individually denoted.

We claim:

1. A method of manufacturing an array plate comprising the steps of:
selecting a plurality of arrays;
transferring the plurality of arrays to a holding plate;
applying adhesive to-a well plate;
transferring the plurality of arrays from the holding plate to the well plate to assemble an array plate.

2. The method of claim 1 wherein the plurality of arrays is selected from at least one wafer.

3. The method of claim 1 wherein the holding plate may be constructed by a plurality of subplates.

4. The method of claim 1 wherein the plurality of arrays may be selected from a plurality of wafers so as to assemble any combination of arrays into a single array plate.

5. The method of claim 1 wherein the adhesive is distributed into a glue stamper with the use of a glue manifold before applying the adhesive to the well plate.

6. The method of claim 1 wherein the glue stamper is an elastomeric plate.

7. The method of claim 1 further comprising curing the array plate;

8. The method of claim 1 wherein the adhesive is UV curable.

9. The method of claim 1 wherein the holding plate can be arranged into a 96-chip format.

10. The method of claim 1 wherein the transferring of the plurality of arrays is done with the use of the a pick-up plate.

11. The method of claim 1 wherein the array plate is disassembled for individual pounching an labeling.

12. An array plate comprising:
a plurality of arrays;
a body comprising a plurality of cavities wherein the body is open at two ends;
and wherein the body has a plurality of pockets at the perimeter of each well at one of the ends and wherein the pockets have high borders;
said plurality of arrays transferred to a holding plate;
said plurality of arrays fixedly connected to the body with the use of an adhesive;
wherein the adhesive is applied to the body with the use of glue stamper.

13. The array plate of claim 12 wherein the plurality of arrays is selected from at least one wafer.

14. The array plate of claim 12 wherein the holding plate may be constructed by a plurality of subplates.

15. The array plate of claim 14 wherein the plurality of subplates are molded with thermoplastic materials.

16. The array plate of claim 14 wherein the plurality of subplates are manufactures with snap-in features for assembly.

17. The array plate of claim 12 wherein the adhesive is distributed on the glue stamper with a glue manifold.

18. The array plate of claim 12 wherein the adhesive is UV curable.

19. The array plate of claim 12 wherein the array plate can be arranged into a 96-chip format
